SEQUENCE LISTING (8 sequences)

SEQ ID NO: 1 - DNA, 20 bp, Artificial Sequence
  Forward primer for GAPDH
attgtcagcaatgcatcctg

SEQ ID NO: 2 - DNA, 20 bp, Artificial Sequence
  Reverse primer for GAPDH
atggactgtggtcatgagcc

SEQ ID NO: 3 - DNA, 20 bp, Artificial Sequence
  Forward primer for CD36
gatcggaactgtgggctcat

SEQ ID NO: 4 - DNA, 24 bp, Artificial Sequence
  Reverse primer for CD36
ggttccttcttcaaggacaacttc

SEQ ID NO: 5 - DNA, 20 bp, Artificial Sequence
  Forward primer for HMG-CoA Reductase
caggatgcagcacagaatgt

SEQ ID NO: 6 - DNA, 20 bp, Artificial Sequence
  Reverse primer for HMG-CoA Reductase
ctttgcatgctccttgaaca

SEQ ID NO: 7 - DNA, 21 bp, Artificial Sequence
  Forward primer for TNF-alpha
ccaccacgctcttctgtctac

SEQ ID NO: 8 - DNA, 20 bp, Artificial Sequence
  Reverse primer for TNF-alpha
agggtctgggccatagaact

(19) United States
(12) Patent Application Publication
(10) Pub. No.: US 2023/0172221 A1
(43) Pub. Date: Jun. 8, 2023

(54) METHOD FOR THE PRODUCTION OF TRADITIONAL KEFIR

(71) Applicants: The Governors of the University of Alberta, Edmonton (CA); Teagasc, The Agriculture and Food Development Authority, Carlow (IE)

(72) Inventors: Benjamin WILLING, Edmonton (CA); Benjamin BOURRIE, Edmonton (CA); Paul COTTER, Fermoy (IE)

(73) Assignees: The Governors of the University of Alberta, Edmonton (CA); Teagasc, The Agriculture and Food Development Authority, Carlow (IE)

(21) Appl. No.: 17/435,666

(22) PCT Filed: Mar. 3, 2020
(86) PCT No.: PCT/CA2020/050283

§ 371 Date: Sep. 1, 2021

Related U.S. Application Data
(60) Provisional application No. 62/813,380, filed on Mar. 4, 2019.

(51) Int. Cl. A23C 9/127

(52) U.S. Cl. CPC A23C 9/127

(57) ABSTRACT

A method for preparing a kefir product involves providing bacterial strains selected from Acetobacter, Leuconostoc, Lactococcus, or Lactobacillus; providing yeast strains selected from Pichia, Saccharomyces, Kazachstania, or Kluyveromyces; adding the bacterial and yeast strains to milk to form a mixture; and allowing the mixture to ferment to yield the kefir product.

### CROSS-REFERENCE TO RELATED APPLICATIONS

This application claims the benefit of U.S. Provisional Pat. Application Serial No. 62/813,380, filed Mar. 4, 2019, the entirety of which is incorporated herein by reference (where permitted).

### FIELD OF THE INVENTION

The present invention relates to a method for preparing and using kefir.

### BACKGROUND OF THE INVENTION

Kefir is a complex fermented dairy product with the ability to confer various health benefits that have been ascribed to whole kefir, kefir microorganisms, lactic acid and/or exopolysaccharides. Such benefits include serum and plasma cholesterol lowering abilities, angiotensin-converting enzyme inhibition, improved cardiac function, antimicrobial activity, tumor suppression, increased speed of wound healing, modulation of the immune system including the alleviation of allergy and asthma, and an ability to improve non-alcoholic fatty liver disease and obesity.

Kefir is traditionally produced through the symbiotic fermentation of milk by lactic acid bacteria and yeasts contained within an exopolysaccharide and protein complex called a kefir grain. However, the use of kefir grains allows for only small scale production of kefir and is not commercially viable for large scale production due to multiple factors including, for example, the need to store and maintain the necessary volume of kefir grains, and the significant differences existing between the microbial composition of individual examples of kefir which may impact the final flavor development and fermentation by-products.

The impact of these microbial differences extends to the ability of kefir to improve circulating cholesterol levels and markers of non-alcoholic fatty liver disease in obese mice, and traditional kefir is better able to improve these phenotypes than a commercial example (Bourrie et al., 2018). This is especially important given the number of commercially available products labelled as kefir that do not contain the microorganisms described as core members of traditional kefir microbial communities, and thus lack the desired health benefits. For example, commercial examples do not typically contain acetic acid bacteria which are ubiquitous among traditional kefirs (Dobson et al., 2011; Marsh et al., 2013; Walsh et al., 2016). While such products may contain Leuconostoc and Lactococcus similar to those found in traditional kefir, the Lactobacillus species contained in many commercial examples are different than those found in kefir grains and grain fermented milk. This is especially important as Lactobacillus kefiranofaciens and L. kefiri, both species unique to kefir, have been shown to have beneficial effects on host health (Carasi et al., 2015; Kim et al., 2017; Chen et al., 2013; Chen et al., 2012). Kefiran, an exopolysaccharide produced by L. kefiranofaciens, has also proven beneficial in vivo (Vinderola et al., 2006; Maeda et al., 2004; Hamet et al., 2016). Another major difference between traditional kefir and commercial products is the lack of a complex yeast community in many commercial products. While some commercial kefir may contain a single species of Saccharomyces, traditional kefir generally contains Saccharomyces cerevisiae, Pichia fermentans, Kazachstania unispora, Kluyveromyces marxianus and K. lactis as well as a multitude of other yeast species at lower levels (Marsh et al., 2013).

Little is known about how interactions between organisms in a complex community impact the ability of fermented foods to improve host metabolic health. As described above, commercial kefirs are produced using different microbial communities and processes than traditional kefir, and specifically do not contain fungal species. Fungal components of traditional fermented foods are especially overlooked in this regard.

Accordingly, there is a need for an improved method of producing kefir.

### SUMMARY OF THE INVENTION

The present invention relates to a method for preparing and using kefir. In one aspect, the invention comprises a method for preparing a kefir product comprising the steps of:
a) providing one or more bacterial strains selected from Acetobacter, Leuconostoc, Lactococcus, or Lactobacillus;
b) providing one or more yeast strains selected from Pichia, Saccharomyces, Kazachstania, or Kluyveromyces;
c) adding the bacterial strains and the yeast strains to milk to form a mixture; and
d) allowing the mixture to ferment to yield the kefir product.

In one embodiment, the bacterial strains comprise at least one Acetobacter species, at least one Leuconostoc species, at least one Lactococcus species, and at least two Lactobacillus species. In one embodiment, the bacterial strains comprise Acetobacter pasteurianus, Leuconostoc mesenteroides, Lactococcus lactis, Lactobacillus kefiranofaciens, and Lactobacillus kefiri. In one embodiment, the concentration comprises 10⁴ CFU/mL of milk of the bacterial strains.

In one embodiment, the yeast strains comprise at least one Pichia species, at least one Saccharomyces species, at least one Kazachstania species, and at least one Kluyveromyces species. In one embodiment, the yeast strains comprise Pichia fermentans, Saccharomyces cerevisiae, Kazachstania unispora, and Kluyveromyces marxianus. In one embodiment, the concentration comprises 10³ CFU/mL of milk of yeast strains.

In one embodiment, the milk comprises pasteurized milk. In one embodiment, the milk comprises at least 2% fat. In one embodiment, fermentation is conducted at room temperature for at least twenty hours.

In one embodiment, the kefir product comprises 10⁸ CFU/mL of milk for bacteria and 10⁶ CFU/mL of milk for yeast. In one embodiment, the kefir product exhibits cholesterol-reducing activity and liver triglyceride-reducing activity.

In another aspect, the invention comprises a kefir product formed by the above method.

In another aspect, the invention comprises a method of treating, preventing, or ameliorating a disease or disorder in a subject, comprising administering a kefir product formed by the above method. In one embodiment, the kefir product reduces cholesterol and liver triglycerides.

In yet another aspect, the invention comprises use of a kefir product formed by the above method to treat, prevent, or ameliorate a disease or disorder in a subject.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 is a graph showing weight gain (g) of mice fed high fat diet (HFD) mixed with commercial kefir (COM), traditional kefir (ICK), pitched kefir (Pitch), pitched kefir with no yeast (PNY), or pitched kefir with no Lactobacillus (PNL) for 8 weeks (n=8).

FIG. 2 is a graph showing fecal fungal plate counts for mice fed HFD and COM, ICK, Pitch, PNY, or PNL at 4 weeks. Counts are expressed as log CFU/g. Means that do not share a letter are significantly different (P<0.05) (n=8).

FIGS. 3A-D are graphs showing plasma total cholesterol (FIG. 3A), HDL cholesterol (FIG. 3B), non-HDL cholesterol (FIG. 3C), and HDL/total cholesterol (FIG. 3D) in mice fed HFD and COM, ICK, Pitch, PNY, or PNL. Levels are expressed in mg/dl. Means that do not share a letter are significantly different (P<0.05) (n=8).

FIG. 4 is a graph showing liver total triglycerides in mice fed a high fat diet supplemented with multiple kefir. Levels are expressed as mg/g protein. Means that do not share a letter are significantly different (P<0.05). N=8.

FIGS. 5A-B are graphs showing average size of lipid droplets (FIG. 5A), and histopathology scores (FIG. 5B) of livers from mice fed a high fat diet supplemented with multiple kefir. N=6-8.

FIGS. 6A-D are graphs showing PPARy (FIG. 6A), CD36 (FIG. 6B), HMG-CoA Reductase (FIG. 6C), and TNF_{α} (FIG. 6D) expression levels in the liver, expressed as fold change relative to COM. Means that do not share a letter are significantly different (P<0.05). N=8.

FIGS. 7A-B show PCoA of cecal microbiota separated by Bray Curtis distance matrix (FIG. 7A) and Alpha diversity measures of cecal microbiota (FIG. 7B).

### DETAILED DESCRIPTION OF PREFERRED EMBODIMENTS

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a,” “an,” and “the” include plural referents unless the context clearly dictates otherwise.

The present invention relates to a method for preparing and using kefir. In particular, the method involves “pitching” to yield pitched kefir. As used herein, the term “pitching” refers to the step of adding bacteria, yeast, or both to initiate fermentation. The method involves the use of specific combinations of bacterial and yeast strains for the preparation of pitched kefir and products derived therefrom.

In one aspect, the present invention comprises a method for preparing a kefir product comprising the steps of:
a) providing one or more bacterial strains selected from Acetobacter, Leuconostoc, Lactococcus, or Lactobacillus;
b) providing one or more yeast strains selected from Pichia, Saccharomyces, Kazachstania, or Kluyveromyces;
c) adding the bacterial strains and the yeast strains to milk to form a mixture; and
d) allowing the mixture to ferment to yield the kefir product.

In one embodiment, the bacterial strains comprise at least one Acetobacter species, at least one Leuconostoc species, at least one Lactococcus species, and at least two Lactobacillus species. In one embodiment, the bacterial strains comprise Acetobacter pasteurianus, Leuconostoc mesenteroides, Lactococcus lactis, Lactobacillus kefiranofaciens, and Lactobacillus kefiri. In one embodiment, the concentration comprises 10⁴ CFU/mL of milk of bacterial strains.

In one embodiment, the yeast strains comprise at least one Pichia species, at least one Saccharomyces species, at least one Kazachstania species, and at least one Kluyveromyces species. In one embodiment, the yeast strains comprise Pichia fermentans, Saccharomyces cerevisiae, Kazachstania unispora, and Kluyveromyces marxianus. In one embodiment, the concentration comprises 10³ CFU/mL of milk of yeast strains.

In one embodiment, the milk comprises pasteurized milk. In one embodiment, the milk comprises at least 2% fat. In one embodiment, the mixture of the bacterial strains, yeast strains, and milk may be left to ferment at room temperature for at least twenty hours. In one embodiment, the kefir product comprises 10⁸ CFU/mL of milk for bacteria and 10⁶ CFU/mL of milk for yeast.

In one embodiment, the invention may comprise a method of treating, preventing, or ameliorating a disease or disorder in a subject, comprising administering the kefir formed by the method of the present invention to the subject. As used herein, the terms “treating,” “preventing,” and “ameliorating” refer to interventions performed with the intention of alleviating the symptoms associated with, preventing the development of, or altering the pathology of a disease, disorder or condition. Thus, in various embodiments, the terms may include the prevention (prophylaxis), moderation, reduction, or curing of a disease, disorder or condition at various stages. In various embodiments, therefore, those in need of therapy/treatment may include those already having the disease, disorder or condition and/or those prone to, or at risk of developing, the disease, disorder or condition and/or those in whom the disease, disorder or condition is to be prevented. As used herein, the term “disease” or “disorder” refers to any condition for which the kefir product may have beneficial effects including, but not limited to, serum and plasma cholesterol lowering abilities, angiotensin-converting enzyme inhibition, antimicrobial activity, tumor suppression, increased speed of wound healing, modulation of the immune system including the alleviation of allergy and asthma, and an ability to improve non-alcoholic fatty liver disease and obesity. As used herein, the term “subject” means a human or other vertebrate.

In one embodiment, the kefir product exhibits the ability to reduce cholesterol and liver triglycerides. The kefir product may provide either subjective relief of symptoms or an objectively identifiable improvement as noted by the clinician or other qualified observer.

In the development of the present invention, the inventors have shown kefir grain fermented milk to be more beneficial in improving cholesterol and lipid metabolism in mice than a commercial kefir product. Further, the inventors have developed a kefir product which is better suited to commercial scale-up using bacteria and yeast isolated from a kefir grain shown to improve plasma cholesterol and liver triglyceride levels. An exemplary process for producing kefir involved initially isolating traditional kefir microbes typically present in a kefir grain and combining the isolated microbes using a pitched approach to produce kefir. The method involved using five strains of bacteria and four strains of yeast. The bacteria comprised Acetobacter pasteurianus, Leuconostoc mesenteroides, Lactococcus lactis, Lactobacillus kefiranofaciens, and Lactobacillus kefiri. The yeast comprised Pichia fermentans, Saccharomyces cerevisiae, Kazachstania unispora, and Kluyveromyces marxianus. The health benefits of traditional grain fermented kefir can thus be recapitulated in a commercial process pitched culture kefir by using these microbes that make up the majority of the traditional kefir microbiota.

In order to examine how the microbial composition of kefir impacts its ability to impart health benefits, the inventors also made pitched culture kefir that lacked either the Lactobacillus or yeast population (referred to as PNL or PNY, respectively) while containing all the other organisms present in the pitched kefir. It was found that the microbial composition of the kefir fermentation is an essential component of the ability of kefir to exert positive influence over the host’s metabolism, with both Lactobacillus and yeast populations identified as being necessary to produce these benefits. The ability of these pitched kefir examples to reduce weight gain, plasma cholesterol profiles, and markers of non-alcoholic fatty liver disease in a mouse model of obesity was then compared to both a commercial kefir and traditional kefir made with the grain from which the pitch organisms were isolated. The results of the Examples should be considered in the development of future commercial kefir products and any other functional products that wish to mimic a traditional fermented food product.

Embodiments of the present invention are described in the following Examples, which are set forth to aid in the understanding of the invention, and should not be construed to limit in any way the scope of the invention as defined in the claims which follow thereafter.

### Example 1 - Kefir Grain Sourcing and Kefir Production

Kefir grains were acquired for a previous study (Kim et al., 2017) and fermentation was carried out as described by Quiros (2005). Pitched kefir was prepared by inoculating pasteurized milk (2% fat) with a mixture of microbes consisting of Acetobacter pasteurianus, Lactococcus lactis, Leuconostoc mesenteroides, Lactobacillus kefiri, Lactobacillus kefiranofaciens, Pichia fermentans, Saccharomyces cerevisiae, Kazachstania unispora, and Kluyveromyces marxianus. The bacterial and yeast strains were grown in culture for twenty-four hours at 30° C. and 5% carbon dioxide, with the culture medium for the bacteria being MRS broth and the culture medium for the yeast being YEG broth. Overnight cultures were inoculated at a starting concentration of 10⁴ colony forming units (CFU)/ml of bacteria and 10³ CFU/ml of yeast. Fermentation occurred under the same conditions as grain fermentation. The bacterial culture, yeast culture, and milk mixture was left to ferment at room temperature for twenty hours. The microbial density of the pitched kefir was 2.4±0.7 × 10⁸ for bacteria, and 6.8±2.8 × 10⁶ for yeast. The microbial density of ICK kefir was 3.0±1.0 × 10⁸ for bacteria, and 5.2±2.2 × 10⁶ for yeast. The microbial density of PNL was 1.9±1.0 × 10⁸ and 7.0±2.0 × 10⁶ for bacteria and yeast respectively, while the PNY kefir had a bacterial density of 2.5±0.6 × 10⁸ while having zero yeast present. The commercial kefir used a microbial composition of Lactobacillus lactis, Lb. rhamnosus, Streptococcus diacetylactis, Lb. plantarum, Lb. casei, Saccharomyces florentinus, Leuconostoc mesenteroides subsp. cremoris, Bifidobacterium longum, Bif. breve, Lb. acidophilus, Bif. lactis, and Lb. reuteri, totaling 8.0 × 10⁶ CFU/ml. The ICK kefir grain used in this study was sequenced (Marsh et al., 2013; Walsh et al., 2016), and contains the major bacterial and fungal genera Lactobacillus, Acetobacter, Leuconostoc, Gluconobacter, Kluyveromyces, Kazachstania, and Dekkera, with a multitude of other low abundance genera.

### Example 2 - Animals and Treatments

Forty eight week old wild type C57BL/6 female mice were obtained from Jackson Labs. Mice were allocated into 6 groups (n=8) consisting of HFD + commercial kefir (COM), HFD + traditional kefir (ICK), HFD + pitched kefir, HFD + pitched kefir without the inclusion of Lactobacillus species, and HFD + pitched kefir without the inclusion of yeast species. Mice received a diet consisting of 40% calories from fat supplemented with 1.25% cholesterol by weight (Research Diets D12108C). Mice were housed under conditions as described in Bourrie et al. (2018). Kefir was mixed into the food daily at a ratio of 2 ml kefir to 20 g of food, which equates to approximately ¼ cup of kefir for a human on a 2000 kcal per day diet. Body weights were taken weekly for the duration of the study and fecal samples were collected weekly for the first 4 weeks of kefir treatment. After 8 weeks, the animals were sacrificed and tissues collected, snap-frozen, and stored at -80° C. until further analysis.

### Example 3 - pH and Bile Tolerance

Screens were conducted through incubation of isolates at a concentration of 10⁶ cells/ml for 4 hours at 30° C. at a pH of 2.5. Bile screens were conducted in 3% oxgall at 10⁶ cells/ml and incubated at 30° C. for 6 hours. After incubation, isolates were plated and growth was compared to a control.

### Example 4 - Fungal Fecal Plating

Fecal samples were collected and weighed prior to being homogenized in phosphate buffered saline. Homogenized samples were then serially diluted and plated on yeast extract glucose chloramphenicol media. Fungal colonies were counted and quantified as CFU/g feces. To determine survival of all kefir yeasts through the tract, DNA was extracted from representative colonies and ITS sequences determined to identify isolates using NCBI BLAST.

### Example 5 - Plasma Cholesterol Measurements

Plasma total cholesterol and high-density lipoprotein (HDL) were determined as described in Bourrie et al. (2018). Non-HDL cholesterol was determined by subtracting HDL cholesterol from total cholesterol.

### Example 6 - Liver Triglyceride Analysis

Liver lipids were extracted and triglycerides were quantified as described in Bourrie et al. (2018).

### Example 7 - Liver Histopathology

Liver tissue was cut and fixed in 10% neutralized formalin buffer for downstream histological analysis. All histological assessments were performed by a single investigator who was blinded to treatment. As manual measurement and counting of vacuoles can be error-prone, hepatocyte of zone 2 according to Rappaport were assessed using an operator-interactive, semi-automated method for quantification of data as previously reported (Amella et al., 2008). The parameters measured from H&E stained sections were the variation of area, perimeter, and width of the vacuoles as well as the variation of their angle, circularity, and Feret, skewness and kurtosis. The Feret diameter is the longest distance between any two points along the selection boundary.

### Example 8 - Gene Expression

Total RNA was isolated from liver tissue and gene expression analysis was conducted as described in Bourrie et al. (2018). Primers for host genes are listed in Table 1.

TABLE 1

Specific primer sequences used for quantitative real-time PCR.

| | | |
|---|---|---|
| Target Gene | Forward (5′-3′) | Reverse (5′-3′) |
| GAPDH | ATTGTCAGCAATGCATCCTG (SEQ ID NO: 1) | ATGGACTGTGGTCATGAGCC (SEQ ID NO: 2) |
| CD36 | GATCGGAACTGTGGGCTCAT (SEQ ID NO: 3) | GGTTCCTTCTTCAAGGACAACTTC (SEQ ID NO: 4) |
| HMG-CoA Reductase | CAGGATGCAGCACAGAATGT (SEQ ID NO: 5) | CTTTGCATGCTCCTTGAACA (SEQ ID NO: 6) |
| TNFα | CCACCACGCTCTTCTGTCTAC (SEQ ID NO: 7) | AGGGTCTGGGCCATAGAACT (SEQ ID NO: 8) |

GAPDH: Glyceraldehyde 3-phosphate dehydrogenase; CD36: Cluster of differentiation 36; HMG-CoA Reductase: 3-hydroxy-3-methyl-glutaryl-coenzyme A reductase; TNFα: Tumor necrosis factor alpha.

### Example 9 - Microbiota Analyses

Total DNA was extracted from caecal content as described in Willing et al. (2011). 16S rRNA gene amplicon sequencing and data analysis was performed as described in Bourrie et al. (2018).

### Example 10 - Statistical Analyses

Plasma cholesterol, liver triglyceride, and gene expression data was analyzed using Analysis of Variance with Tukey post-hoc for multiple comparisons utilizing the R packages multcompView, ggplot2, plyr, and ImPerm. Effect of treatment on microbiota was determined using analysis of similarities (ANOSIM) while relative abundance from phylum to genus taxonomic levels were determined using the Kruskal-Wallis test.

### Example 11 - Results

Discussed below are results obtained in connection with the experiments of Examples 1-10.

### Kefir Composition Did Not Impact Weight Gain

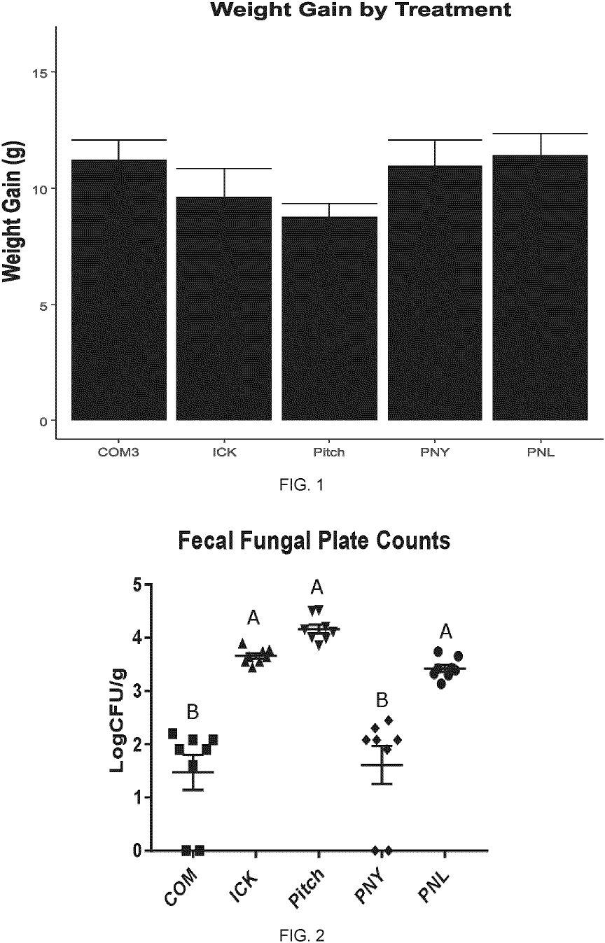

Given the significant differences in microbial communities present in the different kefirs, it was hypothesized that there might be some variability in their ability to impact cholesterol metabolism phenotypes in the presence of a high fat/high cholesterol diet. Since a reduction in weight gain in HFD mice fed a traditional kefir (ICK) had been observed, the inventors further investigated the relative ability of a Pitch culture containing key ICK strains, and PNL and PNY variants thereof, to reduce weight gain in mice fed a high fat diet over an 8 week feeding period, relative to ICK and Commercial kefir controls. After 8 weeks, the COM group had gained the most weight, followed by PNL, PNY, ICK, and Pitch (FIG. 1). While both the ICK and Pitch groups had numerically lower weight gain than the Commercial, PNL, and PNY groups over the 8 weeks, these differences were not significant.

### Kefir Yeast Survived Passage Through the Gastrointestinal Tract

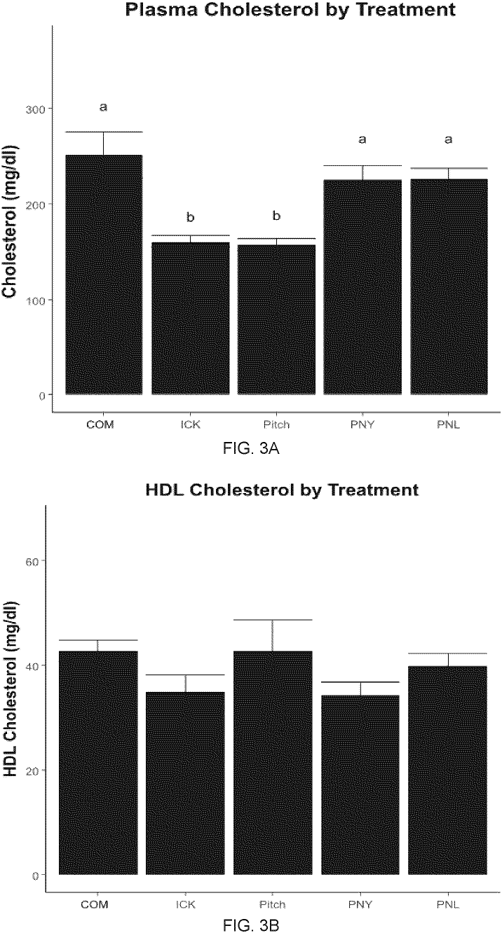

To determine whether traditional kefir yeasts can survive passage through the gastrointestinal tract, fecal samples were taken after four weeks and fungi enumerated by culture. Fungal community composition was assessed by ITS sequencing. Following kefir feeding, the ICK, Pitch, and PNL mice had significantly higher levels of fecal fungal colonies than both the commercial and PNY fed mice, with approximately a 2 log difference being observed (FIG. 2). Additionally, the faecal-derived colonies from each of the ICK, Pitch, and PNL-treatment groups were made up of representatives of each of the major species of yeast present in the kefir (Pichia fermentans, Saccharomyces cerevisiae, Kazachstania unispora, and Kluyveromyces marxianus), indicating that these fungi are able to survive and potentially colonize the gastrointestinal tract. All colonies isolated from the COM and PNY groups belonged to the genus Rhizopus.

TABLE 2

pH, Bile tolerance, and isolation of yeast used in pitched culture kefir.

| | | | | | | |
|---|---|---|---|---|---|---|
| Yeast Species | pH Tolerant | Bile Tolerant | Recovered from COM Fed Mice | Recovered From ICK Fed Mice | Recovered From Pitch Fed Mice | Recovered From PNY Fed Mice |
| Pichia fermentans | +++ | +++ | - | + | + | - |
| Saccharomyces cerevisiae | +++ | +++ | - | + | + | - |
| Kazachstania unispora | +++ | +++ | - | + | + | - |
| Kluyveromyces marxianus | +++ | +++ | - | + | + | - |

+++ = >75% survival in pH or bile tolerance testing. + = successful isolation, - = no successful isolation.

These results suggest that fungi contained in fermented foods may impact host metabolism, and that fermented foods can impact the host mycobiota and improve metabolic health. Additionally, kefir made in a commercial manner with organisms naturally found in TK can recapitulate the health benefits of TK and outperform a widely available commercial example. Most importantly, there is potential to improve existing commercial kefir through the inclusion of traditional kefir organisms, specifically yeast and fungi.

### Certain Kefir Improved Plasma Cholesterol Levels and Profiles

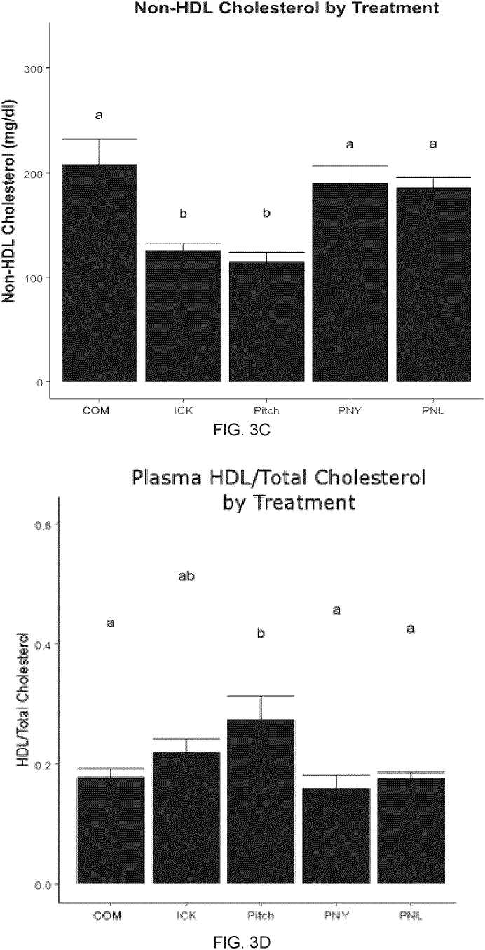

To determine if kefir composition impacted cholesterol metabolism, total plasma cholesterol, HDL, and non-HDL cholesterol levels were analyzed, and the HDL/total cholesterol ratio was calculated. Both the ICK and Pitch groups had similar total cholesterol levels, which were lower than those observed among the COM, PNL, and PNY animals (P<0.05, FIG. 3A). The same pattern between treatments was observed for plasma non-HDL cholesterol; however, plasma HDL cholesterol levels were not significantly different between groups (FIGS. 3B-C). Additionally, the Pitch treated group showed improved HDL:total cholesterol ratios when compared to the COM, PNL, and PNY treated groups (P<0.05, FIG. 3D).

These results appear to be in line with the inventors’ findings (Bourrie et al., 2018) showing that traditional kefir can outperform a commercial example in improving cholesterol profiles in a mouse model of obesity, while also highlighting the importance of the kefir microbial makeup in the health benefits associated with kefir. This is especially important as elevated circulating cholesterol levels are indicative of an increased risk of metabolic syndrome and cardiovascular disease (Despres et al., 2006). Additionally, functional food products are becoming more popular among the public as a means to improve metabolic health, and these results indicate that the microbial composition of these products needs to be considered when evaluating their health benefits.

### Traditional and Pitched Kefir Improved Liver Triglyceride Levels

Another potential disease state associated with obesity and hyperlipidemia is non-alcoholic fatty liver disease (NAFLD), a significant risk factor for the development of steatosis and liver cancer, which is increasing in prevalence worldwide and is threatening to reach epidemic levels (Looma et al., 2013; Duan et al., 2014). One common indicator of NAFLD and non-alcoholic steatohepatitis (NASH) is the level of triglycerides in the liver (Angulo, 2002).

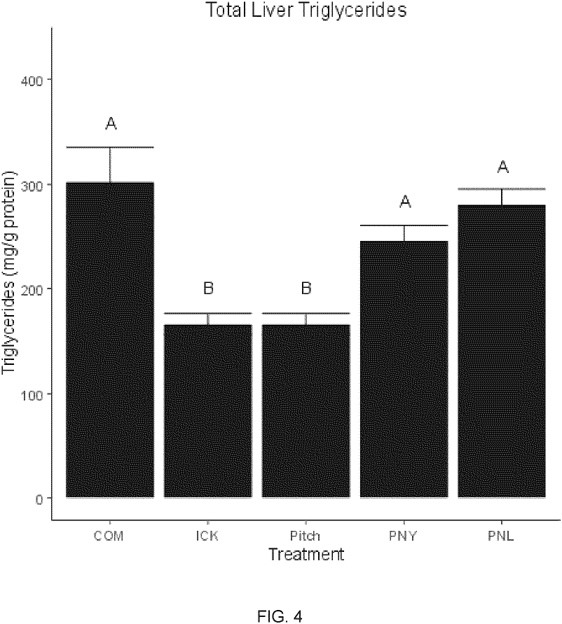

Triglycerides in the liver were measured in order to determine if kefir composition plays a role in protection against NAFLD. Concurrent with the changes observed in plasma cholesterol, ICK and Pitch mice exhibited significantly decreased liver triglycerides when compared to Com, PNY, and PNL mice (FIG. 4). As liver triglyceride levels have been associated with expression levels of certain genes, the inventors examined how PPARy and CD36 expression was altered by kefir treatment as both of these genes have been shown to have increased expression levels when liver triglycerides are increased (Inoue et al., 2005; Dyck et al., 2007; Malaguarnera et al., 2009; Min et al., 2012). Hepatic PPARγ expression was significantly reduced in Pitch mice when compared to PNY and PNL mice and showed a trend to be lower when compared to COM mice, while ICK mice trended towards decreased expression when compared to PNY mice. CD36 expression was also altered, with both ICK and Pitch showing a trend to be lower than PNY and PNL. Additionally, HMG-CoA reductase expression has been shown to correlate with NAFLD and NASH (Min et al., 2012), which may help to further explain the differences observed in liver triglycerides. Without being bound by any theory, these changes together may point to an ability of specific kefirs to alter host lipid metabolism in the liver, leading to a decrease in the hyperlipidemia commonly associated with obesity.

### Kefir Microbiota Did Not Impact Liver Histopathology

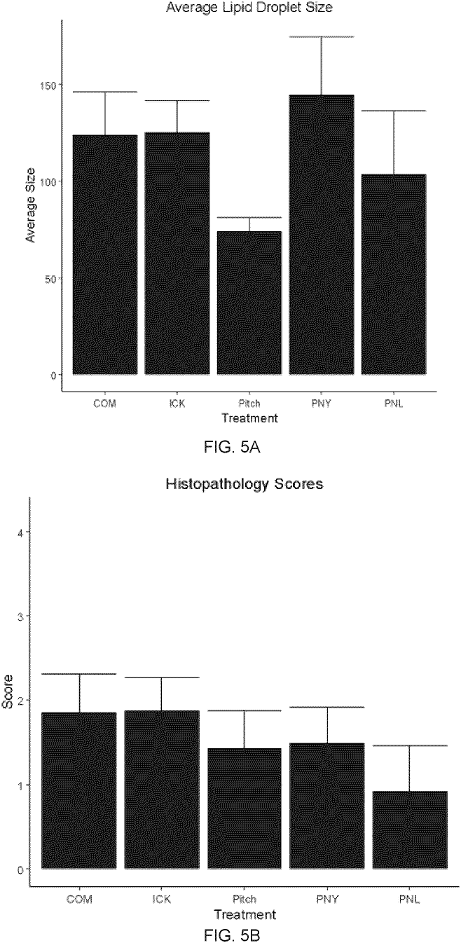

As increased liver triglycerides have been associated with the development of NAFLD and NASH, the inventors examined the average size of lipid droplets in the liver and assigned histological scores for the degree of steatohepatitis. Average size of lipid droplets in the liver was lowest in the Pitch mice, followed by PNL mice, COM, ICK, and finally PNY fed mice; however, there were no statistically significant differences between the groups (FIG. 5A). Histopathological scoring showed no significant differences between groups (FIG. 5B).

This is in contrast to the inventors’ findings related to liver triglyceride levels and lipid metabolism in the liver, which pointed towards certain kefirs being protective against NASH. Without being bound by any theory, the lack of a correlation between liver triglyceride levels and lipid droplet size in the liver may simply be due to an increased deposition of triglycerides which are not present in large lipid droplets. Histopathological scoring showed a distinct lack of a trend and also exhibited extremely high variation. This may be due to a lack of differences observed in the expression levels of the inflammatory cytokine TNFα in the liver as TNFα has been shown to be important in the development of NASH (Crespo et al., 2001; Takahashi et al., 2012). Additionally, recent work has shown the importance of IL-1β and the activation of the NLRP3 inflammasome in the development of steatohepatitis in mice (Miura et al., 2010; Mridha et al., 2017), while previous work failed to find any differences in intestinal expression of the NLRP3 inflammasome markers IL-1β or IL-18 in mice fed kefir on a high fat diet (Bourrie et al., 2018). This lack of an anti-inflammatory effect of kefir may explain the similarities in histopathology scoring between the treatment groups.

### Kefir Composition is a Factor in Improving Host Lipid Metabolism but Not Inflammatory Markers

To determine how different kefir was able to alter circulating cholesterol levels in mice, the expression of each of PPARγ, CD36, and HMG-CoA reductase was measured in the liver. HMG-CoA reductase is an especially important component of cholesterol homeostasis as it is the rate limiting enzyme in the biosynthesis of cholesterol. HMG-CoA reductase inhibitors have been utilized to treat hypercholesterolemia (Grundy, 1988; Reihner et al., 1990).

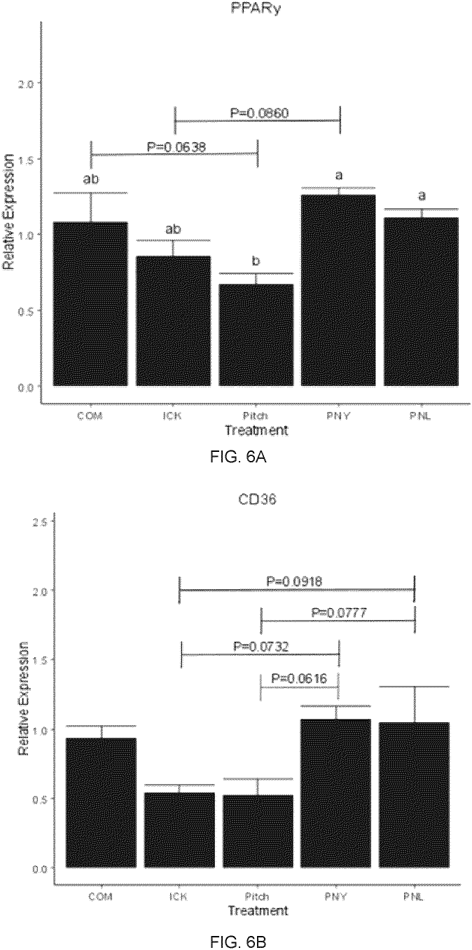

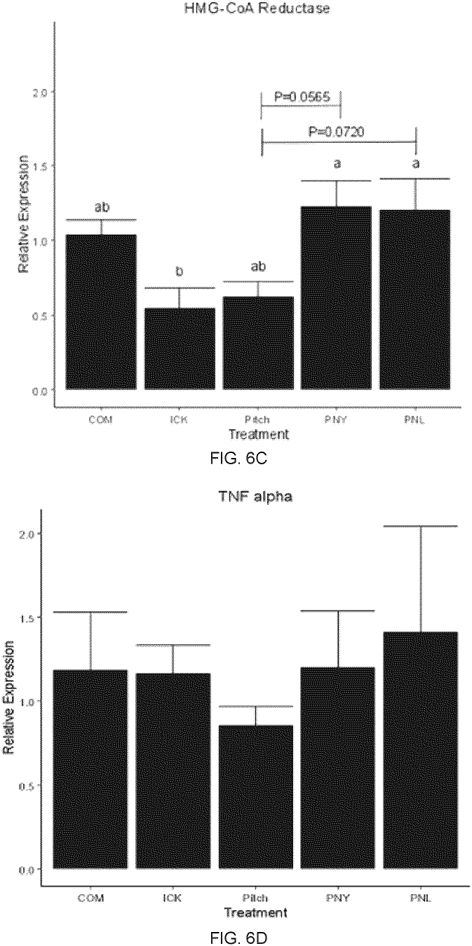

PPARγ expression was significantly lower in the Pitch group when compared to the PNL and PNY (P<0.05, FIG. 6A), while there was a trend for Pitch to be lower than the COM group. ICK did not have significantly lower expression than any of the other groups; however, there was a trend for ICK to be lower than PNL. CD36 showed similar patterns to PPARγ; however, none of the differences in expression levels were significant. PNY and PNL groups showed a trend to have higher expression than both ICK and Pitch (FIG. 6B).

HMG-CoA reductase followed a similar pattern to PPARγ, with ICK having significantly reduced expression compared to PNY and PNL, while Pitch showed a trend to be lower than both PNY and PNL (FIG. 6C). In contrast to the alterations to expression levels of cholesterol related genes in the liver, TNFα expression was not significantly changed by any of the kefir treatments (FIG. 6D). While not significantly different between groups, the pattern of HMG-CoA reductase expression was consistent with differences in plasma cholesterol. Without being bound by any theory, these differences in gene expression may explain the reduction in plasma cholesterol levels observed in the ICK and Pitch groups as expression levels of HMG-CoA reductase in the liver have been found to contribute to increased circulating cholesterol (Chmielewski et al., 2003; Wu et al., 2013).

### Microbiota Composition Analysis

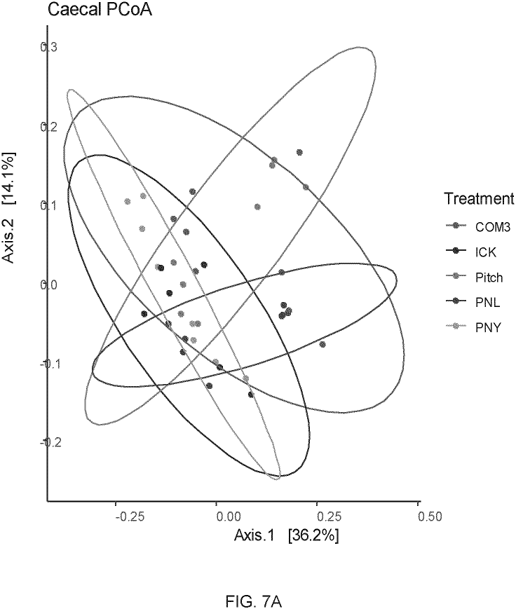

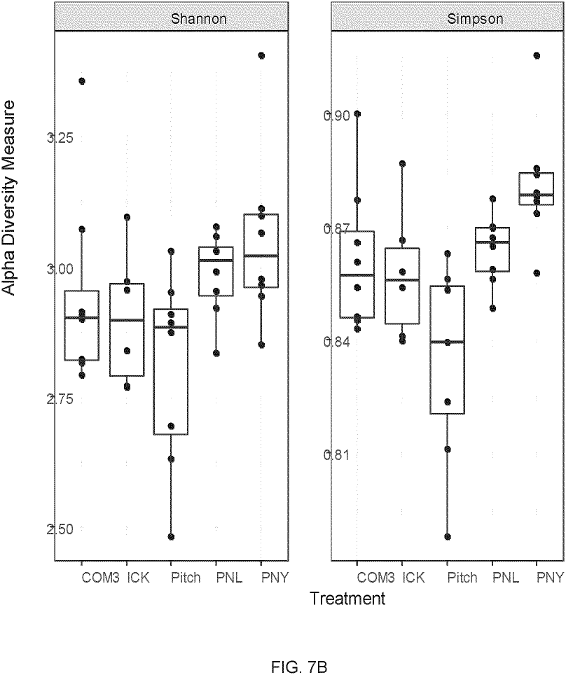

The gastrointestinal microbiota plays an important part in the development of obesity associated metabolic disorders (Everard et al., 2013; Gérard, 2016; Rosenbaum et al., 2015). Given this, and the fact that kefir is generally regarded as a health promoting beverage with beneficial effects on the gut, the bacterial composition of the cecal microbiota was examined following 8 weeks of HFD feeding supplemented with kefir. Beta-diversity of day 56 caecal microbiota was compared using a Bray Curtis distance matrix and visualized with PCoA (FIG. 7A). ADONIS analysis showed a significant effect of treatment (P<0.05). There was minimal separation of groups on the PCoA, with the COM group clustering somewhat separately from the grain fermented and pitched culture kefir fed groups. Without being bound by any theory, this was likely due to the non-detection of Leuconostoc in the gut of commercial mice while each of the other groups contained reads assigned to this genus, although at relatively low abundance (0.0209% to 0.00039% relative abundance). Outside of this genus, there were no other significantly different genera between groups.

This is interesting as the commercial kefir is marketed to contain live cultures of Leuconostoc and suggest that the strain present in this commercial product is less adept at surviving passage through the gastrointestinal tract than those contained in the freshly fermented kefir. It was also notable that, despite containing live yeast culture in the form of Saccharomyces florentinus, the commercial kefir fed mice presented significantly lower levels of fecal fungal colonies when compared to ICK, Pitch, and PNL fed mice. Furthermore, there were no S. florentinus identified from the yeast isolated from COM mice. Without being bound by any theory, the lack of dramatic differences in the gut microbiota of the different groups, as assessed via 16S taxonomic sequencing, suggests that the mechanism of action of kefir is not tied to large scale microbial changes in the gut and is instead dependent on more subtle changes in composition, changes to the microbiome on a functional metabolic level or fermentation products present in the kefir acting directly on the host. Alpha diversity was measured using both the Shannon and Simpson indices and was not significantly different among groups (FIG. 7B).

As previously stated, one possible reason for the differential effects of the different kefirs used in these studies is a difference in the fermentation products generated during the fermentation of each individual kefir. Interestingly, the removal of both yeast and Lactobacillus from the pitched kefir fermentation resulted in the loss of a beneficial impact on cholesterol metabolism. This may point to a relationship between the yeast component and Lactobacilli present in the kefir fermentation which results in the production of a metabolite that is unable to be produced when one group is missing. This hypothesis is supported by recent work identifying interactions between lactobacilli and Saccharomyces species in various fermentations (Mendes et al., 2013; Stadie et al., 2013; Ponomarova et al., 2017) with some of these interactions being shown to be strain dependent. In fact, metabolic by-products of kefir fermentation, such as small peptides and the exopolysaccharide kefiran, have been identified to have potentially positive effects on cholesterol metabolism in the past (Chen et al., 2016; Uchida et al., 2010; Maeda et al., 2005; Maeda et al., 2004; Tung et al., 2017). These studies indicate that there is a possibility that the absence or lowering of a specific metabolic by-product or products could result in a drastically different effect of the kefir in question on the host, once again highlighting the importance of microbial composition in the ability of kefir to benefit host health. Furthermore, the fact that similar fecal yeast counts were obtained for Pitched and PNL suggests that the yeast survival in the tract may not be important to the mechanism. This is in contrast to previous studies which have shown that kefir yeast can lower plasma cholesterol in animal models (Liu et al., 2012; Yoshida et al., 2005); however, these studies utilized pure cultures or cell components of yeast which may explain these differences.

These studies examine how specific alterations to the microbial composition of kefir impacts host health and lipid metabolism in an in vivo model of diet induced obesity. Both the grain fermented ICK kefir and a lab produced commercial process kefir (Pitch) utilizing organisms isolated from ICK lowered plasma total cholesterol, non-HDL cholesterol, and liver triglyceride levels when compared to a widely available commercial kefir as well as lab produced kefir that lacked either the Lactobacillus or yeast population. Without being bound by any theory, these greater impacts were likely due to an alteration of the host cholesterol and lipid metabolism in the liver based on observed changes to gene expression profiles. The results show that, although many commercial kefirs have microbes of the same genera as those present in traditional kefir, the exact species and perhaps even strain of these species may be essential to the health benefits observed in previous studies utilizing traditionally fermented kefir. Further, the health benefits of traditional kefir can be recapitulated utilizing traditional kefir organisms in a process of producing kefir using pitched cultures, indicating a potential important consideration in the future development of large scale kefir production. Additionally, these studies highlight the importance of microbial composition and interactions in functional fermented foods and indicate that a failure to accurately replicate or retain key microbes present in such foods can have detrimental effects on the ability of the functional food to exert a positive influence on the host.

### REFERENCES

All publications mentioned are incorporated herein by reference (where permitted) to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Amella C, Cappello F, Kahl P, Fritsch H, Lozanoff S, Sergi C. Spatial and temporal dynamics of innervation during the development of fetal human pancreas. Neuroscience. 2008; 154:1477-87.

Angulo P. Nonacoholic Fatty Liver Disease. N Engl J Med. 2002;346:1221-31.

Bourrie BCT, Willing BP, Cotter PD. The microbiota and health promoting characteristics of the fermented beverage kefir. Front Microbiol. 2016;7:1-17.

Bourrie BCT, Cotter PD, Willing BP. Traditional kefir reduces weight gain and improves plasma and liver lipid profiles more successfully than a commercial equivalent in a mouse model of obesity. J Funct Foods [Internet]. Elsevier; 2018;46:29-37.

Carasi P, Racedo SM, Jacquot C, Romanin DE, Serradell MA, Urdaci MC. Impact of Kefir Derived Lactobacillus kefiri on the Mucosal Immune Response and Gut Microbiota. J Immunol Res [Internet]. 2015;2015:e361604.

Chen HL, Tsai TC, Tsai YC, Liao JW, Yen CC, Chen CM. Kefir peptides prevent high-fructose corn syrup-induced non-alcoholic fatty liver disease in a murine model by modulation of inflammation and the JAK2 signaling pathway. Nutr Diabetes [Internet]. Nature Publishing Group; 2016;6:e237.

Chen H-L, Tung Y-T, Tsai C-L, Lai C-W, Lai Z-L, Tsai H-C, Lin Y-L, Wang C-H, Chen C-M. Kefir improves fatty liver syndrome by inhibiting the lipogenesis pathway in leptin-deficient ob/ob knockout mice. Int J Obes. 2013;38.

Chen YP, Chen MJ. Effects of Lactobacillus kefiranofaciens M1 isolated from kefir grains on germ-free mice. PLoS One [Internet]. Elsevier; 2013;8:7467-77.

Chen YP, Hsiao PJ, Hong WS, Dai TY, Chen MJ. Lactobacillus kefiranofaciens M1 isolated from milk kefir grains ameliorates experimental colitis in vitro and in vivo. J Dairy Sci [Internet]. Elsevier; 2012;95:63-74.

Chmielewski M, Sucajtys E, Swierczynski J, Rutkowski B, Bogusɬawski W. Contribution of increased HMG-CoA reductase gene expression to hypercholesterolemia in experimental chronic renal failure. Mol Cell Biochem. 2003;246:187-91.

Choi J-W, Kang HW, Lim W-C, Kim M-K, Lee I-Y, Cho H-Y. Kefir prevented excess fat accumulation in diet-induced obese mice. Biosci Biotechnol Biochem [Internet]. Taylor & Francis; 2017;8451:1-8.

Crespo J, Cayón A, Fernandez-Gil P, Hernández-Guerra M, Mayorga M, Dominguez-Díez A, Fernández-Escalante JC, Pons-Romero F. Gene expression of tumor necrosis factor α and TNF-receptors, p55 and p75, in nonalcoholic steatohepatitis patients. Hepatology. 2001;34:1158-63.

Després J-P, Lemieux I. Abdominal obesity and metabolic syndrome. Nature [Internet]. Nature Publishing Group; 2006 [cited 2017 Jul 8];444:881-7.

Dobson A, O’Sullivan O, Cotter PD, Ross P, Hill C. High-throughput sequence-based analysis of the bacterial composition of kefir and an associated kefir grain. FEMS Microbiol Lett. 2011 ;320:56-62.

Duan XY, Zhang L, Fan JG, Qiao L. NAFLD leads to liver cancer: Do we have sufficient evidence? Cancer Lett [Internet]. Elsevier Ireland Ltd; 2014;345:230-4.

Dyck JRB, Koonen DPY, Vance DE, Soltys C-LM, Febbraio M, Young ME, Jacobs RL, Ong H. Increased Hepatic CD36 Expression Contributes to Dyslipidemia Associated With Diet-Induced Obesity. Diabetes. 2007;56:2863-71.

Everard A, Cani PD. Diabetes, obesity and gut microbiota. Best Pract Res Clin Gastroenterol. 2013;27:73-83.

Fathi Y, Ghodrati N, Zibaeenezhad M-J, Faghih S. Kefir drink causes a significant yet similar improvement in serum lipid profile, compared with low-fat milk, in a dairy-rich diet in overweight or obese premenopausal women: A randomized controlled trial. J Clin Lipidol. 2016.

Garrote GL, Abraham AG, Rumbo M. Is lactate an undervalued functional component of fermented food products? Front Microbiol. 2015;6:1-5.

Gerard P. Gut microbiota and obesity. Cell Mol Life Sci. 2016;73:147-62.

Grundy SM. HMG-CoA Reductase Inhibitors for Treatment of Hypercholesterolemia. N Engl J Med. 1988;319:24-33.

Hamet MF, Medrano M, Perez PF, Abraham AG. Oral administration of kefiran exerts a bifidogenic effect on BALB/c mice intestinal microbiota. Benef Microbes. 2016.

Hutkins RW (Robert W. Microbiology and technology of fermented foods [Internet]. [cited 2019 Apr 27]. 473 p.

Inoue M, Ohtake T, Motomura W, Takahashi N, Hosoki Y, Miyoshi S, Suzuki Y, Saito H, Kohgo Y, Okumura T. Increased expression of PPARy in high fat diet-induced liver steatosis in mice. Biochem Biophys Res Commun. 2005;336:215-22.

Kim D-H, Jeong D, Kang I-B, Kim H, Song K-Y, Seo K-H. Dual function of Lactobacillus kefiri DH5 in preventing high-fat-diet-induced obesity: direct reduction of cholesterol and upregulation of PPARα in adipose tissue. Mol Nutr Food Res [Internet]. 2017; 1700252.

Kim DH, Kim H, Jeong D, Kang IB, Chon JW, Kim HS, Song KY, Seo KH. Kefir alleviates obesity and hepatic steatosis in high-fat diet-fed mice by modulation of gut microbiota and mycobiota: targeted and untargeted community analysis with correlation of biomarkers. J Nutr Biochem [Internet]. Elsevier Inc.; 2017;44:35-43.

Liu H, Xie YH, Xiong LX, Dong RT, Pan CL, Teng GX, Zhang HX. Effect and Mechanism of Cholesterol-Lowering by Kluyveromyces from Tibetan Kefir. Adv Mater Res [Internet]. 2012;343-344:1290-8.

Liu J-R, Wang S-Y, Chen M-J, Chen H-L, Yueh P-Y, Lin C-W. Hypocholesterolaemic effects of milk-kefir and soyamilk-kefir in cholesterol-fed hamsters. Br J Nutr [Internet]. 2006;95:939-46.

Loomba R, Sanyal AJ. The global NAFLD epidemic. Nat Rev Gastroenterol Hepatol [Internet]. Nature Publishing Group; 2013;10:686-90.

Maeda H, Zhu X, Suzuki S, Suzuki K, Kitamura S. Structural Characterization and Biological Activities of an Exopolysaccharide Kefiran Produced by Lactobacillus kefiranofaciens WT-2BT. J Agric Food Chem [Internet]. 2004;52:5533-8.

Maeda H, Mizumoto H, Suzuki M, Tsuji K. Effects of Kefiran-Feeding on Fecal Cholesterol Excretion, Hepatic Injury and Intestinal Histamine Concentration in Rats. Biosci Microflora. 2005;24:35-40.

Maeda H, Zhu X, Omura K, Suzuki S, Kitamura S. Effects of an exopolysaccharide (kefiran) on lipids, blood pressure, blood glucose, and constipation. BioFactors. 2004;22:197-200.

Malaguarnera M, Di Rosa M, Nicoletti F, Malaguarnera L. Molecular mechanisms involved in NAFLD progression. J Mol Med. 2009;87:679-95.

Marco ML, Heeney D, Binda S, Cifelli CJ, Cotter PD, Foligne B, Ganzle M, Kort R, Pasin G, Pihlanto A, et al. Health benefits of fermented foods: microbiota and beyond. Curr Opin Biotechnol. 2017;44:94-102.

Marsh AJ, O’Sullivan O, Hill C, Ross RP, Cotter PD. Sequencing-Based Analysis of the Bacterial and Fungal Composition of Kefir Grains and Milks from Multiple Sources. PLoS One. 2013;8.

Mendes F, Sieuwerts S, de Hulster E, Almering MJH, Luttik MAH, Pronk JT, Smid EJ, Bron PA, Daran-Lapujadea P. Transcriptome-based characterization of interactions between Saccharomyces cerevisiae and Lactobacillus delbrueckii subsp. bulgaricus in lactose-grown chemostat cocultures. Appl Environ Microbiol. 2013;79:5949-61.

Metchnikoff E. The Prolongation of Life. Putnam; 1908.

Min HK, Kapoor A, Fuchs M, Mirshahi F, Zhou H, Maher J, Kellum J, Warnick R, Contos MJ, Sanyal AJ. Increased hepatic synthesis and dysregulation of cholesterol metabolism is associated with the severity of nonalcoholic fatty liver disease. Cell Metab [Internet]. Elsevier Inc.; 2012;15:665-74.

Miura K, Kodama Y, Inokuchi S, Schnabl B, Aoyama T, Ohnishi H, Olefsky JM, Brenner DA, Seki E. Toll-Like Receptor 9 Promotes Steatohepatitis by Induction of Interleukin-1β in Mice. Gastroenterology [Internet]. Elsevier Inc.; 2010;139:323-334.e7.

Mridha AR, Wree A, Robertson AAB, Yeh MM, Johnson CD, Van Rooyen DM, Haczeyni F, Teoh NCH, Savard C, loannou GN, et al. NLRP3 inflammasome blockade reduces liver inflammation and fibrosis in experimental NASH in mice. J Hepatol [Internet]. European Association for the Study of the Liver; 2017;66: 1037-46.

Ostadrahimi A, Taghizadeh A, Mobasseri M. Effect of Probiotic Fermented Milk ( Kefir ) on Glycemic Control and Lipid Profile In Type 2 Diabetic Patients: A Randomized. Iran J Public Health. 2015;44:228-37.

Ponomarova O, Sevin D, Mülleder M, Zirngibl K, Bulya K. Yeast creates a stable niche for symbiotic lactic acid bacteria through nitrogen overflow. Cell Syst. 2017;1-13.

Quiros A. Angiotensin-Converting Enzyme Inhibitory Activity of Peptides Derived from Caprine Kefir. J Dairy Sci. 2005;88:3480-7.

Reihner E, Rudling M, Stahlberg D, Berglund L, Ewerth S, Bjorkhem I, Einarsson K, Angelin B. Influence of Pravastatin, a Specific Inhibitor of HMG-CoA Reductase, on Hepatic Metabolism of Cholesterol. N Engl J Med. 1990;323:224-8.

Rosenbaum M, Knight R, Leibel RL. The gut microbiota in human energy homeostasis and obesity. Trends Endocrinol Metab [Internet]. Elsevier Ltd; 2015;26:493-501.

Silva-Cutini MA, Almeida SA, Nascimento AM, Abreu GR, Bissoli NS, Lenz D, Endringer DC, Brasil GA, Lima EM, Biancardi VC, et al. Long-term treatment with kefir probiotics ameliorates cardiac function in spontaneously hypertensive rats. J Nutr Biochem [Internet]. Elsevier Inc.; 2019;66:79-85.

Stadie J, Gulitz A, Ehrmann MA, Vogel RF. Metabolic activity and symbiotic interactions of lactic acid bacteria and yeasts isolated from water kefir. Food Microbiol [Internet]. Elsevier Ltd; 2013;35:92-8.

Takahashi Y, Soejima Y, Fukusato T. Animal models of nonalcoholic fatty liver disease/ nonalcoholic steatohepatitis. World J Gastroenterol. 2012; 18:2300-8.

Tung Y-T, Chen H-L, Wu H-S, Ho M-H, Chong K-Y, Chen C-M. Kefir Peptides Prevent Hyperlipidemia and Obesity in High Fat Diet-Induced Obese Rats via Lipid Metabolism Modulation. Mol Nutr Food Res [Internet]. 2017;1700505.

Uchida M, Ishii I, Inoue C, Akisato Y, Watanabe K, Hosoyama S, Toida T, Ariyoshi N, Kitada M. Kefiran Reduces Atherosclerosis in Rabbits Fed a High Cholesterol Diet. J AtherosclerThromb. 2010; 17:980-8.

Vinderola CG, Duarte J, Thangavel D, Perdigón G, Farnworth E, Matar C. Immunomodulating capacity of kefir. J Dairy Res. 2005;72:195-202.

Vinderola G, Perdigón G, Duarte J, Farnworth E, Matar C. Effects of the oral administration of the exopolysaccharide produced by Lactobacillus kefiranofaciens on the gut mucosal immunity. Cytokine. 2006;36:254-60.

Walsh AM, Crispie F, Kilcawley K, O’Sullivan O, O’Sullivan MG, Claesson MJ, Cotter PD. Microbial Succession and Flavor Production in the Fermented Dairy Beverage Kefir. mSystems [Internet]. 2016;1:e00052-16.

Willing BP, Vacharaksa A, Croxen M, Thanachayanont T, Finlay BB. Altering host resistance to infections through microbial transplantation. PLoS One. 2011;6:2-10.

Wu N, Sarna LK, Hwang S, Zhu Q, Wang P, Siow YL, K O. Regulation of Hmg-Coa Reductase in Diet-Induced Non-Alcoholic Fatty Liver Disease. Can J Cardiol. 2013;29:S378.

Yoshida Y, Yokoi W, Ohishi K, Ito M, Naito E, Sawada H. Effects of the cell wall of Kluyveromyces marxianus YIT 8292 on the plasma cholesterol and fecal sterol excretion in rats fed on a high-cholesterol diet. Biosci Biotechnol Biochem. 2005;69:714-23.

As will be apparent to those skilled in the art, various modifications, adaptations and variations of the foregoing specific disclosure can be made without departing from the scope of the invention claimed herein.

1. A method for preparing a kefir product comprising the steps of:
a) providing one or more bacterial strains selected from Acetobacter, Leuconostoc, Lactococcus, or Lactobacillus;
b) providing one or more yeast strains selected from Pichia, Saccharomyces, Kazachstania, or Kluyveromyces;
c) adding the bacterial strains and the yeast strains to milk to form a mixture; and
d) allowing the mixture to ferment to yield the kefir product.

2. The method of claim 1, wherein the bacterial strains comprise at least one Acetobacter species, at least one Leuconostoc species, at least one Lactococcus species, and at least two Lactobacillus species.

3. The method of claim 2, wherein the bacterial strains comprise Acetobacter pasteurianus, Leuconostoc mesenteroides, Lactococcus lactis, Lactobacillus kefiranofaciens, and Lactobacillus kefiri.

4. The method of claim 3, wherein the concentration comprises 10⁴ CFU/mL of milk of the bacterial strains.

5. The method of claim 1, wherein the yeast strains comprise at least one Pichia species, at least one Saccharomyces species, at least one Kazachstania species, and at least one Kluyveromyces species.

6. The method of claim 5, wherein the yeast strains comprise Pichia fermentans, Saccharomyces cerevisiae, Kazachstania unispora, and Kluyveromyces marxianus.

7. The method of claim 6, wherein the concentration comprises 10³ CFU/mL of milk of yeast strains.

8. The method of claim 1, wherein the milk comprises pasteurized milk.

9. The method of claim 8, wherein the milk comprises at least 2% fat.

10. The method of claim 1, wherein fermentation is conducted at room temperature for at least twenty hours.

11. The method of claim 1, wherein the kefir product comprises 10⁸ CFU/mL of milk for bacteria and 10⁶ CFU/mL of milk for yeast.

12. The method of claim 1, wherein the kefir product exhibits cholesterol-reducing activity and liver triglyceride-reducing activity.

13. A kefir product formed by the method of claim 1 .

14. A method of treating, preventing, or ameliorating a disease or disorder in a subject, comprising administering a kefir product formed by the method of claim 1.

15. The method of claim 14, wherein the kefir product reduces cholesterol and liver triglycerides.

16. Use of a kefir product formed by the method of claim 1 to treat, prevent, or ameliorate a disease or disorder in a subject.
